(19) United States
(12) Patent Application Publication
(10) Pub. No.: US 2007/0032420 A1
(43) Pub. Date: Feb. 8, 2007

(54) Treating diabetes with glucagon-like peptide-1 secretagogues

(75) Inventors: David Polidori, Rancho Sante Fe, CA (US); Scott Siler, Hayward, CA (US)

Correspondence Address: ENTELOS, INC.;c/o FOLEY & LARDNER LLP, 1530 PAGE MILL RD., PALO ALTO, CA, 94304, US

(73) Assignee: Entelos, Inc.

(21) Appl. No.: 11/352,455

(22) Filed: Feb. 9, 2006

Related U.S. Application Data
(60) Provisional application No. 60/651,739, filed on Feb. 9, 2005.

(51) Int. Cl. A61K 38/30; A61K 38/04

(52) U.S. Cl. 514/12; 514/19

(57) ABSTRACT

In general this invention can be viewed as encompassing novel methods of treating diabetes and insulin resistance. The inventors have made the discovery that increasing secretion of endogenous glucagon-like peptide-1 (GLP-1) in combination with inhibiting the activity of dipeptidyl peptidase I (DPP-IV) can have a significant impact on hyperglycemia and insulin secretion in subjects suffering from diabetes and/or insulin resistance. Further the invention encompasses methods of identifying subjects having elevated secretion of GLP-1, methods of assessing sensitivity to a GLP-1 secretagogue, and methods of treating diabetes in these subjects by administering a GLP-1 secretagogue to alleviate at least one symptom of diabetes.

### CROSS-REFERENCE TO RELATED PATENT APPLICATIONS

This application claims the benefit of U.S. Provisional Application No. 60/651,739, filed 9 Feb. 2005, incorporated herein by reference in its entirety.

### FIELD OF THE INVENTION

This invention relates to methods of treating diabetes and insulin resistance in a subject.

### BACKGROUND OF THE INVENTION

Glucagon-like peptide-1 (GLP-1) is an endogenous hormone that possesses antidiabetogenic activity. GLP-1 is released by the L cells of the small intestine upon stimulation with nutrients, particularly in the duodenum (Schirra, et al. J Clin Invest. 97:92-103 (1996)) and ileum (Layeret, al. Dig Dis Sci. 40:1074-82 (1995)). GLP-1 stimulates insulin release in the presence of hyperglycemia (Kjems, et al. Diabetes 52:380-6 (2003); Fritsche, et al. Eur J Clin Invest. 30:411-8 (2000); Brandt, et al. Am J Physiol Endocrinol Metab. 281:E242-7 (2001); Quddusi, et al. Diabetes Care 26:791-8 (2003)). Moreover, it appears that this stimulatory ability is retained in patients with type 2 diabetes (Elahi, et al. Regul Pept. 51:63-74 (1994)). In addition to its actions as an insulin secretagogue, GLP-1 also reduces glucagon excursions (Kolterman, et al. J Clin Endocrinol Metab. 88:3082-9 (2003)), inhibits gastric emptying (Willms, et al. J Clin Endocrinol Metab. 81:327-32 (1996); Meier, et al. J Clin Endocrinol Metab. 88:2719-25 (2003)), and reduces food intake (Flint, et al. J Clin Invest. 101:515-20 (1998)). Taken together, these effects act to reduce hyperglycemia.

GLP-1 is inactivated by the exopeptidase dipeptidyl peptidase IV (DPP-IV) (Deacon, et al. J Clin Endocrinol Metab. 80:952-7 (1995)). DPP-IV acts on GLP-1 and other substrates (including glucose-dependent insulinotropic peptide, vasoactive intestinal polypeptide, neuropeptide Y, and many cytokines) to remove amino acids from the amino terminus of the protein (Mentlein Regul Pept. 85:9-24 (1999)). Removal of amino terminal amino acids renders GLP-1 unable to properly bind and activate its receptor. The effective half-life of intact, active GLP-1 is approximately 90 seconds in vivo (Deacon, et al. J Clin Endocrinol Metab. 80:952-7 (1995); Vilsboll, et al. J Clin Endocrinol Metab. 88:220-4 (2003)).

Elevation of active GLP-1 levels is emerging as a viable approach for treating type 2 diabetes (D'Alessio and Vahl, Am J Physiol Endocrinol Metab. 286:E882-90 (2004); Deacon, Diabetes 53:2181-9 (2004)). There are several means of increasing active GLP-1 levels that are currently under development in the pharmaceutical and biotechnology community, with each having specific attributes and drawbacks. One of the challenges to therapeutically elevating active GLP-1 is rapid inactivation by DPP-IV (Deacon, et al. J Clin Endocrinol Metab. 80:952-7 (1995); Vilsboll, et al. J Clin Endocrinol Metab. 88:220-4 (2003)). Subcutaneous infusion of GLP-1 has been demonstrated to be quite effective in normalizing glucose levels in subjects (Meneilly, et al. Diabetes Care 26:2835-41 (2003); Zander, et al. Lancet 359:824-30 (2002); Toft-Nielsen, et al. Diabetes Care 22:1137-43 (1999); Nauck, et al. Diabetologia 39:1546-53 (1996)). However, constant infusion (as opposed to bolus injection) is required to provide adequate levels of active GLP-1. This approach requires patients to wear a pump, which is cumbersome and poses increased risk of infection (Rivera-Alsina, and Willis. Diabetes Care 7:75-6 (1984)). Bolus injection of chemically or genetically modified GLP-1 has also been attempted. In this class of therapy, GLP-1 is modified such that it resists the actions of DPP-IV, but retains the ability to serve as an agonist for the GLP-1 receptor. One drawback of these compounds is compromised agonist activity. Several of these molecules are in various stages of development (Baggio, et al. Diabetes 53:2492-500 (2004)), with Degn et al. (Diabetes 53:1187-94 (2004)) recently reporting data from a clinical study with liraglutide. To date no modified GLP-1 molecule has been developed that both resists inactivation by DPP-IV and maintains high anti-hyperglycemic activity.

GLP-1 receptor agonists are also being developed as therapeutic approaches for type 2 diabetes. Similar to the modified GLP-1 agents, these molecules interact with the GLP-1 receptor, but not DPP-IV. Perhaps the best known representative of this class is exendin-IV, which was first discovered in the saliva of the Gila monster (Goke, et al. J Biol. Chem. 268:19650-5 (1993); Egan, et al. Am J Physiol Endocrinol Metab. 284:E1072-9 (2003)). Exendin-IV is a peptide that is similar in composition to GLP-1, but lacks the amino acid sequence required to serve as a substrate for DPP-IV (Doyle, et al. Regul Pept. 114:153-8 (2003)). As exendin-IV is a peptide, subcutaneous injection is required for drug delivery. Some subjects who have been given exendin-IV (Exanatide) have reported experiencing nausea (Egan, et al. Am J Physiol Endocrinol Metab. 284:E1072-9 (2003)).

The final category of agents that elevate active GLP-1 levels are the DPP-IV inhibitors (Deacon, et al. Expert Opin Investig Drugs 13:1091-102 (2004)). These agents reduce the ability of DPP-IV to exert its peptidase actions on GLP-1 (and other molecules), thereby increasing active GLP-1 levels. DPP-IV inhibitors also restrict the conversion of many other molecules, including those that participate in immune function (Mentlein Regul Pept. 85:9-24 (1999)). A primary concern in the development of these agents is the risk that is posed by altering the function of key peptides of the immune system. Moreover, early clinical studies with DPP-IV inhibitors have utilized substantial inhibition of DPP-IV (>90% inhibited over 24 hrs) to lower glucose levels just enough to be considered efficacious (Ahren, et al. Diabetes Care 25:869-75 (2002); Ahren, et al. J Clin Endocrinol Metab. 89:2078-84 (2004)). Indeed, Ahren et al. reported a relatively high percentage of subjects reporting various adverse events associated with altered immune function after receiving the DPP-IV inhibitor LAF237 for four weeks (Ahren, et al. J Clin Endocrinol Metab. 89:2078-84 (2004)).

One approach to elevating active GLP-1 levels in subjects with type 2 diabetes is to increase the endogenous secretion of GLP-1. Several dietary components are potent secretagogues, including oleic acid (Rocca, et al. Endocrinology 142:1148-55 (2001)), other fatty acids (Thomsen, et al. Am J Clin Nutr. 69:1135-43 (1999)), and carbohydrates (Schirra, et al. J Clin Invest. 97:92-103 (1996)). Yasuda et al. (Biochem Biophys Res Commun. 298:779-84 (2002)) also recently demonstrated that biguanides like metformin also appear to increase GLP-1 secretion. Elevating secretion of GLP-1 alone suffers from the same obstacle as other therapies do, inasmuch as endogenously produced GLP-1 will be converted to its inactive form by DPP-IV. According to the present invention, combining enhanced GLP-1 production with DPP-IV inhibition will increase efficacy and reduce the incidence of adverse events. Less DPP-IV inhibition would be required to elevate active GLP-1 levels if it were combined with increased GLP-1 release. The concomitant reduction in exopeptidase conversion of non-GLP-1 peptides would also be reduced, resulting in fewer alterations in normal immune and endocrine function.

### SUMMARY OF THE INVENTION

One aspect of the invention provides methods of alleviating at least one symptom of diabetes comprising concurrently administering a therapeutically effective amount of a glucagon-like peptide-1 (GLP-1) secretagogue and a therapeutically effective amount of an inhibitor of dipeptidyl peptidase IV (DPP-IV) activity to a subject having diabetes. In a preferred embodiment, the subject has type 2 diabetes. Preferably, the GLP-1 secretagogue increases basal GLP-1 levels by at least two-fold, more preferably by at least three-fold. Preferably the DPP-IV inhibitor decreases DPP-IV activity by at least 40%. The DPP-IV inhibitor also preferably decreases DPP-IV activity by less than 100%, more preferably by no greater than 60%. The symptom of diabetes may be, inter alia, elevated plasma glycosylated hemoglobin (HbA1c), elevated blood glucose concentration, or decreased insulin concentration. In a preferred embodiment, the subject's HbA1c decreases by at least 1.0% (absolute difference), more preferably by at least 1.2% and most preferably by at least 1.7%. In another preferred embodiment, the subjects' twenty-four hour average blood glucose level decreases by at least 21% (relative difference), more preferably by at least 28% and most preferably by at least 32%. In preferred embodiments, the DPP-IV inhibitor is selected from the group consisting of valine pyrrolidide, isoleucine-thiazolidide, 1-[[(3-hydroxy-1-adamantyl)amino]acetyl]-2-cyano-(S)-pyrrolidine (LAF237), 1-[[[2-[(5-cyanopyridin-2-yl)amino]ethyl]amino]acetyl]-2-cyano-(S)-pyrrolidine (NVP DPP728), and (2S)-1-([2S]-2′-amino-3′,3′-dimethylbutanoyl)-pyrrolidine-2-carbonitrile (FE999011). The GLP-1 secretagogue preferably is administered enterally, parenterally, or transdermally. In a preferred embodiment, the GLP-1 secretagogue is administered via the lumen of the intestines.

Another aspect of the invention provides methods of alleviating at least one symptom of insulin resistance comprising concurrently administering a therapeutically effective amount of a glucagon-like peptide-1 (GLP-1) secretagogue and a therapeutically effective amount of an inhibitor of dipeptidyl peptidase IV (DPP-IV) activity to an insulin-resistant subject.

An aspect of the invention provides methods of alleviating at least one symptom of diabetes in a diabetic subject having elevated secretion of GLP-1, said method comprising administering a therapeutically effective amount of a glucagon-like peptide-1 (GLP-1) secretagogue. In a preferred embodiment, the subject's HbA1c decreases by at least 1.0% (absolute difference), more preferably by at least 1.6% and most preferably by at least 1.9%. In another preferred embodiment, the subjects' twenty-four hour average blood glucose level decreases by at least 18% (relative difference), more preferably by at least 27% and most preferably by at least 35%. Preferably, the GLP-1 secretagogue increases basal GLP-1 levels by at least two-fold, more preferably by at least three-fold. The GLP-1 secretagogue also preferably increases postprandial GLP-1 levels by at least two-fold, more preferably by at least three-fold.

One aspect of the invention provides methods of assessing elevated secretion of GLP-1 in a subject comprising (a) measuring a fasting GLP-1 level in the subject after a fast, (b) orally administering about 50 g to about 100 g of glucose to the subject, (c) measuring a stimulated GLP-1 level about 20 to about 90 minutes after orally administering the glucose, and (d) diagnosing the subject as having elevated secretion of GLP-1 if the stimulated GLP-1 level is greater than two-fold the fasting GLP-1 level.

Another aspect of the invention provides methods of assessing sensitivity to GLP-1 secretagogue therapy comprising (a) measuring a fasting GLP-1 level in a subject after a fast, (b) orally administering about 50 g to about 100 g of glucose to the subject, (c) measuring a stimulated GLP-1 level about 20 to about 90 minutes after orally administering the glucose, and (d) identifying the subject as sensitive to GLP-1 secretagogue therapy if the stimulated GLP-1 level is greater than two-fold the fasting GLP-1 level.

Yet another aspect of the invention provides methods of manufacturing a drug for use in the treatment of diabetes comprising: (a) identifying a compound as a GLP-1 secretagogue and (b) formulating said compound for concurrent administration to a subject with an inhibitor of dipeptidyl peptidase IV activity. The compound can be identified as a GLP-1 secretagogue, and thereby useful in the treatment of diabetes or insulin resistance, by (i) comparing an amount of GLP-1 secretion in the presence of the compound with an amount of GLP-1 secretion in the absence of the compound; and (ii) identifying the compound as useful in the treatment of diabetes when the amount of GLP-1 secretion in the presence of the compound is at least two-fold greater than the amount of GLP-1 secretion in the absence of the compound.

An aspect of the invention provides a package comprising a GLP-1 secretagogue, an inhibitor of DPP-IV and instructions for concurrently administering the secretagogue and the inhibitor for treating diabetes and/or insulin resistance.

It will be appreciated by one of skill in the art that the embodiments summarized above may be used together in any suitable combination to generate additional embodiments not expressly recited above, and that such embodiments are considered to be part of the present invention.

### BRIEF DESCRIPTION OF THE DRAWINGS

FIG. 1 illustrates DPP-IV activity with administration of DPP-IV inhibitors. Inhibitors were administered to virtual patients at t=0.75 and t=9.75 hours (fifteen minutes prior to the first and final meals of the day). Filled circles (●) denote with the effects of 40% DPP-IV inhibition and filled squares (▪) denote 100% inhibition. DPP-IV activity remains at 100% throughout the day when not inhibited.

FIG. 2 illustrates a linear regression of the reduction in HbA1c against the increase in 24 hour active GLP-1 levels. Each point represents the data from an individual virtual patient undergoing a particular therapeutic intervention. The correlation was strong between the two, with r²=0.84.

### DETAILED DESCRIPTION OF THE PREFERRED EMBODIMENTS

A. Overview

In general this invention can be viewed as encompassing novel methods of treating diabetes and insulin resistance. The inventors have made the discovery that increasing secretion of endogenous glucagon-like peptide-1 (GLP-1) in combination with inhibiting the activity of dipeptidyl peptidase I (DPP-IV) can have a significant impact on hyperglycemia and insulin secretion in subjects suffering from diabetes and/or insulin resistance. Further the invention encompasses methods of identifying subjects having elevated secretion of GLP-1, methods of assessing sensitivity to a GLP-1 secretagogue, and methods of treating diabetes in these subjects by administering a GLP-1 secretagogue to alleviate at least one symptom of diabetes.

B. Definitions

“Administering” means any of the standard methods of administering a pharmaceutical composition known to those skilled in the art. Examples include, but are not limited to enteral, transdermal, intravenous, intramuscular or intraperitoneal administration.

“Concurrent administration” and “concurrently administering” as used herein includes administering a compound capable of increasing GLP-1 secretion and a compound capable of inhibiting DPP-IV activity in admixture, such as, for example, in a pharmaceutical composition or in solution, or as separate compounds, such as, for example, separate pharmaceutical compositions or solutions administered consecutively, simultaneously, or at different times but not so distant in time such that the compound capable of increasing GLP-1 secretion and the compound capable of inhibit DPP-IV activity cannot interact.

The term “drug” refers to a compound of any degree of complexity that can affect a biological system, whether by known or unknown biological mechanisms, and whether or not used therapeutically. Examples of drugs include typical small molecules (molecules having molecular weights of less than 1000 daltons) of research or therapeutic interest; naturally-occurring factors such as endocrine, paracrine, or autocrine factors, antibodies, or factors interacting with cell receptors of any type; intracellular factors such as elements of intracellular signaling pathways; factors isolated from other natural sources; pesticides; herbicides; and insecticides. Drugs can also include, agents used in gene therapy such as DNA and RNA. Also, antibodies, viruses, bacteria, and bioactive agents produced by bacteria and viruses (e.g., toxins) can be considered as drugs. A response to a drug can be a consequence of, for example, drug-mediated changes in the rate of transcription or degradation of one or more species of RNA, drug-mediated changes in the rate or extent of translational or post-translational processing of one or more polypeptides, drug-mediated changes in the rate or extent of degradation of one or more proteins, drug-mediated inhibition or stimulation of action or activity of one or more proteins, and so forth. In some instances, drugs can exert their effects by interacting with a protein. For certain applications, drugs can also include, for example, compositions including more than one drug or compositions including one or more drugs and one or more excipients.

The phrase “elevated secretion of GLP-1” as used herein, refers to the magnitude of increase in GLP-1 levels in response to a standard meal challenge and corresponds to a 50% greater change in GLP-1 levels from basal to peak following a meal challenge as compared with normal diabetic individuals. The phrase “a subject having elevated secretion of GLP-1” refers to a subject that has an increased native level of secretion of GLP-1, such that the subject experiences a 50% greater change in GLP-1 levels from basal to peak following a meal challenge as compared with a normal diabetic individual. Typically, a subject having elevated secretion of GLP-1 will have a GLP-1 after ingesting 50-100 g of glucose that is at least twice the subject's GLP-1 level after fasting.

As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, its use in the therapeutic compositions is contemplated.

The term “subject” refers to any warm-blooded animal, preferably a human. Subjects having diabetes can include, for example, subjects that have been diagnosed with diabetes, subjects that exhibit one or more of the symptoms associated with diabetes, or subjects that are progressing towards or are at risk of developing diabetes.

As used herein, a “therapeutically effective amount” of a drug of the present invention is intended to mean that amount of the compound that will achieve the intended physiological effect, e.g., increased GLP-1 secretion or inhibition of DPP-IV, and thereby cause the regression and palliation of at least one symptom associated with diabetes and/or insulin resistance.

C. Methods of Treatment with GLP-1 Secretagogues

Based on observations of an in silico model providing mathematical representations of multiple macronutrient metabolism, we found that administration of a GLP-1 secretagogue, alone or concurrent with an inhibitor of DPP-IV, will alleviate symptoms of diabetes, especially decreasing glycosylated hemoglobin, decreasing blood glucose levels and increasing insulin levels. These observations also take into account alterations in lipid and amino acid metabolism.

In silico modeling integrates relevant biological data—genomic, proteomic, and physiological—into a computer-based platform to reproduce a system's control principles. Given a set of initial conditions representing a defined disease state, these computer-based models can simulate the system's future biological behavior, a process termed biosimulation. A model similar to that used for the current analysis is described in co-pending U.S. patent application Ser. No. 10/040,373, published 27 Mar. 2003 as U.S.2003-0058245.

The computer model allows a user to simulate a variety of diabetic and pre-diabetic subjects by combining defects in various combinations where those defects have various degrees of severity. This can allow a more effective modeling of the type 2 diabetes population, which is heterogeneous. In other words, diabetes can have a wide range of impairment, some of which can be distinguished clinically. Furthermore, clinically similar diabetics can have differences in their physiology that can be modeled by using different defect combinations. Consequently, the computer model can be used to better understand and classify the real patient population for type 2 diabetes and to anticipate what drug target may work best on certain classes of subjects, thereby improving the design of clinical trials and target prioritization.

In sum, the computer model can enable a researcher, for example, to: (1) simulate the dynamics of hyperglycemia in type 2 diabetes, (2) visualize key metabolic pathways and the feedback within and between these pathways, (3) gain a better understanding of the metabolism and physiology of type 2 diabetes, (4) explore and test hypotheses about the metabolism of normal subjects or those with type 2 diabetes and normal metabolisms, (5) identify and prioritize potential therapeutic targets, (6) identify patient types and their responses to various interventions, and (7) organize knowledge and data that relate to type 2 diabetes.

The computer model is expect to behave in a manner similar to the biological states it represents as closely as possible and can be validated against biological responses of real subjects. The computer model can be validated, for example, with in vitro and in vivo data obtained using reference patterns of the biological state being modeled. The current model was validated using methods substantially similar to those described in co-pending application Ser. No. 10/151,581 entitled “Apparatus and Method for Validating a Computer Model,” published on Dec. 19, 2002 as U.S.2002-0193979.

The potential efficacy of GLP-1 secretagogues as an approach to treating type 2 diabetes was tested in this study. 17 virtual patients were used, the characteristics of which are shown in Table 1. Two distinct subpopulations were incorporated into the study to reflect the variability observed in postprandial GLP-1 excursions in subjects with type 2 diabetes. One subpopulation (n=12) was chosen to reflect the relatively low GLP-1 excursions (57-83% increase over fasted) that are reported by Vilsboll et al. (Diabetes 50:609-13 (2001); J Clin Endocrinol Metab. 88:4897-903 (2003)). Another subpopulation (n=5) of virtual patients was chosen to reflect the relatively high GLP-1 excursions (129% increase over fasted) that have been reported by Ahren et al. (J Clin Endocrinol Metab. 89:2078-84 (2004)). All virtual patients were given a diet that preserved energy balance and contained 55% carbohydrate, 30% fat, and 15% protein.

TABLE 1
Virtual Patient Characteristics

| | | | Fasting Plasma | Fasting Plasma |
|---|---|---|---|---|
| HbA1c | Body Weight | | Glucose | Insulin |
| (%) | (kg) | % Body Fat | (mg/dl) | (μU/ml) |
| 8.6 ± 1 | 85.2 ± 0.6 | 28.6 ± 2.4 | 171.5 ± 29.5 | 17.4 ± 3.7 |

Several different levels of enhanced GLP-1 secretion were simulated for each patient in this study. An increased basal rate of release and an amplified postprandial release rate were each simulated individually and in combination. For the postprandial treatment, the response to the presence of nutrients in the intestines was increased two- or three-fold. The basal rate of release was amplified by increasing the parameter associated with the basal rate of release two- or three-fold. The total simulation time was 90 days.

The primary measurements that were made before and after each treatment were HbA1c (glycosylated hemoglobin), active GLP-1 levels, 24 hour average glucose levels, and 24 hour average insulin levels. To give insight into the relative potency of the insulin secretory stimulation provided by elevated active GLP-1 levels for a given level of glucose, the ratio of 24 hour average insulin to 24 hour average glucose was computed. Additionally, GLP-1 has a profound effect on gastric emptying (Meier, et al. J Clin Endocrinol Metab. 88:2719-25 (2003)), and the results from some simulations indicate that persistently-elevated active GLP-1 levels can cause gastric nutrients to accumulate. When gastric nutrients were greater than 500 g in a virtual patient in the overnight-fasted state undergoing a particular treatment, the results for that virtual patient were not utilized. The reason for this is that the accumulation of nutrients in the stomach in this way would undoubtedly influence behavior (such as food consumption) that would in turn generate different results for that treatment. All values are reported as the mean of the group±the standard deviation for the group.

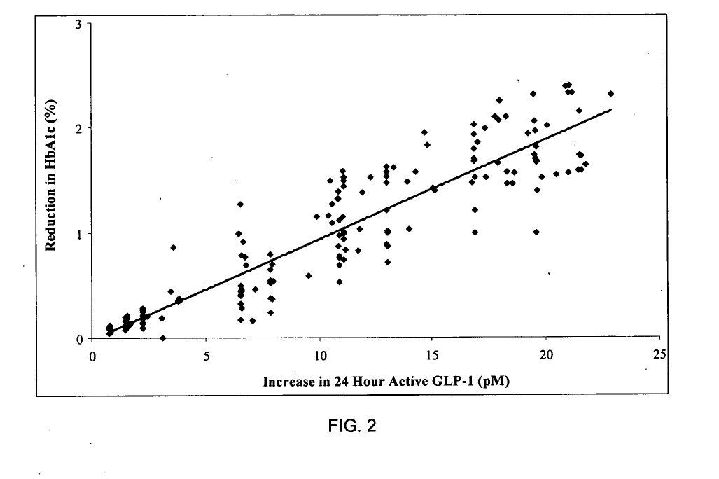

Enhanced GLP-1 release was an effective treatment in all groups of virtual patients, although it should be noted that the degree of increases simulated for this study were relatively high—two- to three-fold over baseline (Table 2). For this study, a treatment was considered efficacious when HbA1c was lowered by at least 1% (absolute difference). Three monotherapies achieved this magnitude of glycemic reduction in all virtual patients: three-fold increase in basal GLP-1 secretion, and two- and three-fold increases in basal and postprandial GLP-1 release. Two- and three-fold increases in postprandial GLP-1 secretion were also efficacious in the subpopulation of subjects with elevated GLP-1 secretion. These results highlight one observation to emerge from this study, that the association between the magnitude of active GLP-1 increase in a virtual patient and the concomitant reduction in HbA1c for that patient is similar for all the therapies considered in this study (FIG. 2). The correlation between the two in this study was strong, with a coefficient of determination (r²) equal to 0.84. Based on this observation, any treatment that seeks to treat type 2 diabetes by altering active GLP-1 levels will have the greatest impact when GLP-1 is raised as high as possible; according to the regression curve, an increase in 24 hour average active GLP-1 levels of 10.7 pM will lead to a reduction of HbA1c of 1% (FIG. 2).

TABLE 2
Results of increased GLP-1 secretion
Treatments

| | | | | | | |
|---|---|---|---|---|---|---|
| | | | | | 2× basal + | 3× basal + |
| | 2× post- | 3× post- | | | 2× post- | 3× post- |
| Pre-trx | prandial | prandial | 2× basal | 3× basal | prandial | prandial |

All Virtual Patients

| | | | | | | | |
|---|---|---|---|---|---|---|---|
| HbA1c (%) | 8.6 ± 1.0 | 8.3 ± 1.1 | 8.0 ± 1.3 | 8.0 ± 1.1 | 7.2 ± 0.9 | 7.6 ± 1.2 | 7.1 ± 0.7 |
| 24 hour average | 223.8 ± 34.7 | 210.2 ± 38.4 | 133.3 ± 45.9 | 199.3 ± 37.9 | 167 ± 33.4 | 184.3 ± 43.8 | 164.4 ± 25.8 |
| glucose (mg/dl) | | | | | | | |
| 24 hour average | 37.1 ± 4.0 | 39.6 ± 4.9 | 41.3 ± 6.3 | 40.8 ± 5.3 | 47.0 ± 6.6 | 43.7 ± 7.4 | 48.0 ± 5.7 |
| insulin (μU/ml) | | | | | | | |
| Ration of 24 hour | 17.0 ± 3.4 | 19.6 ± 5.3 | 22.4 ± 8.7 | 21.5 ± 6.4 | 29.5 ± 9.4 | 25.8 ± 10.9 | 30.0 ± 6.5 |
| insulin to glucose (μU | | | | | | | |
| insulin/mg glucose) | | | | | | | |
| 24 average active GLP-1 (pM) | 8.2 ± 2.0 | 11.0 ± 4.8 | 14.2 ± 8.2 | 16.2 ± 3.9 | 24.0 ± 5.7 | 20.5 ± 8.1 | 24.6 ± 2.3 |

High GLP-1 Secreting Virtual Patients

| | | | | | | | |
|---|---|---|---|---|---|---|---|
| HbA1c (%) | 8.3 ± 1.0 | 7.4 ± 0.9 | 6.7 ± 0.8 | 7.2 ± 0.9 | 6.4 ± 0.7 | 6.4 ± 0.7 | n.d. |
| 24 hour average | 214.3 ± 37.3 | 180.8 ± 30.9 | 152.8 ± 28.0 | 169.1 ± 31.2 | 139.6 ± 25.2 | 140.0 ± 24.7 | n.d |
| glucose (mg/dl) | | | | | | | |
| 24 hour average | 37.2 ± 3.8 | 43.2 ± 3.5 | 47.4 ± 4.1 | 44.6 ± 4.1 | 50.8 ± 6.1 | 50.9 ± 6.1 | n.d. |
| insulin (μU/ml) | | | | | | | |
| Ration of 24 hour | 17.9 ± 4.0 | 24.5 ± 5.1 | 32.1 ± 7.8 | 27.3 ± 6.8 | 37.8 ± 10.5 | 37.7 ± 10.5 | n.d. |
| insulin to glucose (μU | | | | | | | |
| insulin/mg glucose) | | | | | | | |
| 24 average active GLP-1 (pM) | 10.9 ± 0.3 | 17.5 ± 0.2 | 25.4 ± 0.8 | 21.5 ± 0.2 | 31.8 ± 0.6 | 31.6 ± 1.0 | n.d. |

Twenty-four hour average plasma glucose levels were also reduced in response to all treatments. Before any treatment, 24 hour average plasma glucose levels were 223.8±34.7 mg/dl in the total virtual patient population and 214.3±37.3 mg/dl in the subpopulation of virtual patients with high GLP-1. When the postprandial release was increased two-fold, 24 hour average plasma glucose was reduced only 6% to 210.2±38.4 mg/dl in the total virtual patient population and 16% to 180.8±30.9 mg/dl in the subpopulation with elevated GLP-1 levels. A three-fold increase in the postprandial GLP-1 release rate lowered 24 hour glucose 40% and 29% to 133.3±45.9 mg/dl and 152.8±28.0 mg/dl in the total population and the subpopulation, respectively. A two-fold increase in the basal rate of GLP-1 release lowered 24 hour glucose levels 11% to 199.3±37.9 mg/dl in the total virtual patient population and 21% to 169.1±31.2 mg/dl in the subpopulation. When the basal release rate was simulated to be three-fold greater than baseline, 24 hour average glucose levels were reduced 25% to 167.9±33.4 mg/dl and 35% to 139.6±25.2 mg/dl in the total virtual patient population and the subpopulation with high GLP-1, respectively. The combined treatment of two-fold increases in the postprandial and the basal GLP-1 release rates lowered 24 hour glucose levels 18% to 184.3±43.8 mg/dl in the total population and 35% to 140.0±24.7 mg/dl in the subpopulation. Three-fold increases in the postprandial and basal rates of GLP-1 release gave rise to a 27% reduction in 24 hour glucose (to 164.4±25.8 mg/dl) in the total virtual patient population. As indicated above, the results from the subpopulation of virtual patients with high GLP-1 were not appropriate for reporting due to a non-physiologic level of gastric nutrients.

The ratio of the 24 hour average plasma insulin to the 24 hour average plasma glucose was computed for each treatment of this study (Table 2). This was done to better understand the role that the GLP-1 stimulation of insulin release played in reducing glucose for each treatment. The ratio increased 15% (17.0±3.4 μU insulin/mg glucose baseline) and 32% (17.9±4.0 μU insulin/mg glucose baseline) in the total population and 37% and 79% in the subpopulation of virtual patients when postprandial GLP-1 release was increased two- and three-fold, respectively. When the basal rate was increased two-fold, the ratio increased 26% in the total population and 53% in the subpopulation; three-fold increased basal GLP-1 release increased the ratio 74% in the total population and 111% in the subpopulation. Combining two-fold increased basal secretion with two-fold increased postprandial GLP-1 release caused the ratio of 24 hour insulin to 24 hour glucose to increase 52% in the total population and 111% in the subpopulation. Three-fold increases in basal and postprandial GLP-1 release raised the ratio in the total population 76%.

As expected, 24 hour average active GLP-1 levels increased in response to all treatments. Twenty-four hour average active GLP-1 levels were 8.2±2.0 pM in the total virtual patient population and 10.9±0.3 pM in the subpopulation of virtual patients with high GLP-1. Two-fold increases in the postprandial GLP-1 release rate led to a 34% increase (11.0±4.8 pM) in 24 hour GLP-1 levels in the total population and 61% increase (17.5±0.2 pM) in the subpopulation. Three-fold increased postprandial GLP-1 release increased 24 hour active GLP-1 to 14.2±8.2 pM (73% increase) in the total population and 25.4±0.8 pM (133% increase). When the basal release rate was increased two-fold, 24 hour active GLP-1 levels increased 98% to 16.2±3.9 pM in the total population and 97% to 21.5±0.2 pM in the subpopulation. The three-fold increased basal GLP-1 release rate caused 24 hour GLP-1 levels to increase 193% to 24.0±5.7 pM and 192% to 31.8±0.6 pM in the total population and the subpopulation, respectively. The combined two-fold increase in the postprandial and two-fold increased basal GLP-1 release rate elicited 150% (20.5±8.1 pM) and 190% (31.6±1.0 pM) increases in 24 hour active GLP-1 levels in each group. Three-fold increased basal plus postprandial GLP-1 release caused 24 hour GLP-1 to increase 200% to 24.6±2.3 pM in the total virtual patient population.

The efficacy of GLP-1 secretagogue treatment was similar to other treatment approaches aimed at increasing active GLP-1 levels. DPP-IV inhibition with LAF237 for four weeks lowered 24 hour average glucose levels by 16% (Ahren, et al. J Clin Endocrinol Metab. 89:2078-84 (2004)). The cohort of virtual patients in the present study also exhibited a 16% or greater reduction when treated with two- or three-fold increased basal plus postprandial GLP-1 release and three-fold increased basal GLP-1 release. GLP-1 analogs are also used to lower glycemia in subjects with type 2 diabetes. A recent report by Degn et al. observed a 20% decrease in 24 hour average glucose levels when the GLP-1 analog liraglutide was administered for one week in subjects with type 2 diabetes (Degn, et al. Diabetes 53:1187-94 (2004)). Reductions of this magnitude were seen when virtual patients were treated with three-fold increased basal GLP-1 release, and two- or three-fold increased basal plus postprandial release. The GLP-1 receptor agonist Exanatide has also been reported to lower glycemia effectively. Egan et al. reported that HbA1c was reduced from 9.1% to 8.3% after one month of treatment with Exanatide (Egan, et al. Am J Physiol Endocrinol Metab. 284:E1072-9 (2003)). Accounting for the underestimation of the magnitude of HbA1c reduction due to the short duration of the study by Egan et al., several of the treatments simulated in this report are comparable to the results seen with Exanatide treatment. HbA1c was reduced by 1-1.5% when the virtual patients were treated with three-fold increased basal GLP-1 release and two- or three-fold increased basal plus postprandial release.

GLP-1 is an effective antidiabetogenic agent due to its actions in increasing insulin secretion (Kjems, et al. Diabetes 52:380-6 (2003); Fritsche, et al. Eur J Clin Invest. 30:411-8 (2000); Brandt, et al. Am J Physiol Endocrinol Metab. 281:E242-7 (2001); Quddusi, et al. Diabetes Care 26:791-8 (2003)), decreasing glucagon release (Kolterman, et al. J Clin Endocrinol Metab. 88:3082-9 (2003)), and inhibiting gastric emptying (Willms, et al. J Clin Endocrinol Metab. 81:327-32 (1996); Meier, et al. J Clin Endocrinol Metab. 88:2719-25 (2003)). In this study, insulin secretion was elevated, particularly when compared to the prevailing glucose levels. Also, restrictions in gastric emptying lowered postprandial glucose and glucagon excursions (data not shown). When active GLP-1 was persistently raised to very high levels, the gastric contents of the virtual patients did not completely empty between the dinner meal and the subsequent breakfast meal in some of the virtual patients. The results from these virtual patients were not included when summarizing data, as this result indicated a disturbance in normal physiology. Although not included in the current model, retention of gastric nutrients would likely influence food intake. GLP-1 has been demonstrated to mediate reductions in food intake (Flint, et al. J Clin Invest. 101:515-20 (1998)), and the retention of gastric nutrients may help explain this observation. Also, several of the treatments that serve to increase active GLP-1 levels report that some subjects experience nausea (Degn, et al. Diabetes 53:1187-94 (2004); Egan, et al. Am J Physiol Endocrinol Metab. 284:E1072-9 (2003)). It is possible that the retention of gastric nutrients caused by the increased levels of active GLP-1 could be the cause of this relatively high rate of nausea with GLP-1 elevation.

D. Methods of Treatment by Concurrent Administration of GLP-1 Secretagogues and DPP-IV Inhibitors

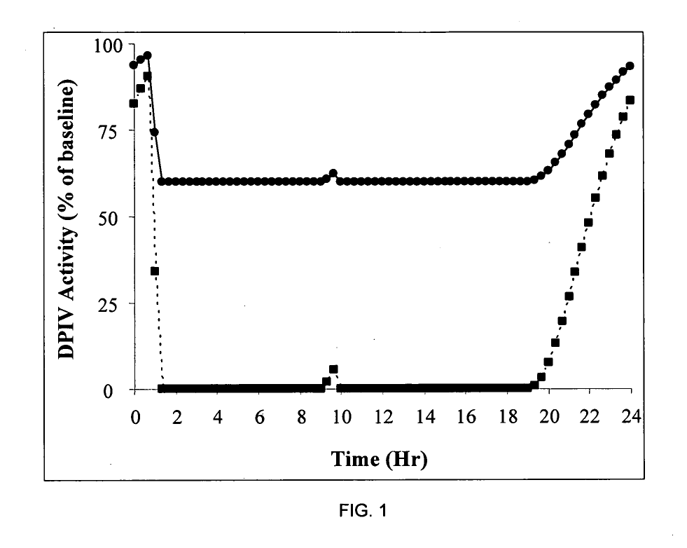

An aspect of the invention provides methods of alleviating at least one symptom of diabetes comprising concurrently administering a therapeutically effective amount of a glucagon-like peptide-1 (GLP-1) secretagogue and a therapeutically effective amount of an inhibitor of dipeptidyl peptidase IV (DPP-IV) activity to a subject having diabetes. The potential efficacy of treatment of diabetes, especially type 2 diabetes, with concurrent administration of GLP-1 secretagogues and DPP-IV inhibitors was examined. The same 17 virtual patients, described above in the analysis of GLP-1 secretagogue monotherapy, were used for this study. Two levels of enhanced GLP-1 secretion (two-fold and three-fold increase in basal rate of GLP-1 release) and a single level of decreased GLP-1 inactivation (40% DPP-IV inhibition) were simulated for each patient. In addition, two different degrees of DPP-IV inhibitor monotherapy were simulated. DPP-IV was inhibited 40% throughout the majority of the day in one treatment arm and 100% in the other arm (FIG. 1).

The results for all treatments are listed in Table 3 and summarized below. Glycosylated hemoglobin was reduced in response to several treatments. HbA1c was 8.6±1.0% for the total virtual patient population and 8.3±1.0% in the high GLP-1 secreting population prior to treatment. When a two-fold increased basal release rate was combined with 40% inhibition of DPP-IV, HbA1c fell to 7.4±1.0% in the total virtual patient population and 6.6±0.8% in the subpopulation. A three-fold increase in the basal release rate plus 40% inhibition of DPP-IV gave HbA1c of 7.0±0.7% in the total population, with the subpopulation of virtual patients with high GLP-1 again accumulating a non-physiologic level of gastric nutrients (rendering the results invalid). When DPP-IV was inhibited 40% alone, HbA1c fell to only 8.4±1.0% in the total population and 7.9±0.9% in the subpopulation. DPP-IV inhibition of 100% lowered HbA1c to 7.2±0.9% in the total virtual patient population and 6.5±0.8% in the subpopulation with high GLP-1.

TABLE 3
Results of Increase GLP-1 Secretion and/or DPP-IV Inhibition

| | | | | |
|---|---|---|---|---|
| | 2× basal + | 3× basal + | | |
| Pre-trx | 40% DPP-IV | 40% DPP-IV | 40% DPP-IV | 100% DPP-IV |

All Virtual Patients

| | | | | | |
|---|---|---|---|---|---|
| HbA1c (%) | 8.6 ± 1.0 | 7.4 ± 1.0 | 7.0 ± 0.7 | 8.4 ± 1.0 | 7.2 ± 0.9 |
| 24 hour average | 223.8 ± 34.7 | 175.9 ± 35.9 | 161.0 ± 24.1 | 213.3 ± 33.9 | 170.3 ± 32.7 |
| glucose (mg/dl) | | | | | |
| 24 hour average | 37.1 ± 4.0 | 45.3 ± 6.4 | 48.9 ± 5.5 | 39.0 ± 4.4 | 46.4 ± 5.8 |
| insulin (μU/ml) | | | | | |
| Ratio of 24 hour | 17.0 ± 3.4 | 27.2 ± 8.5 | 31.1 ± 6.6 | 18.7 ± 3.8 | 28.5 ± 7.7 |
| insulin to glucose (μU | | | | | |
| insulin/mg glucose) | | | | | |
| 24 average active GLP-1 (pM) | 8.2 ± 2.0 | 21.5 ± 5.3 | 26.7 ± 0.8 | 11.0 ± 2.7 | 21.7 ± 5.3 |

High GLP-1 Secreting Virtual Patients

| | | | | | |
|---|---|---|---|---|---|
| HbA1c (%) | 8.3 ± 1.0 | 6.6 ± 0.8 | n.d. | 7.9 ± 0.9 | 6.5 ± 0.8 |
| 24 hour average | 214.3 ± 37.3 | 146.0 ± 27.0 | n.d. | 198.0 ± 32.7 | 144.5 ± 25.6 |
| glucose (mg/dl) | | | | | |
| 24 hour average | 37.2 ± 3.8 | 49.0 ± 5.2 | n.d. | 39.8 ± 3.8 | 48.8 ± 4.4 |
| insulin (μU/ml) | | | | | |
| Ratio of 24 hour | 17.9 ± 4.0 | 34.8 ± 9.2 | n.d. | 20.6 ± 4.1 | 34.9 ± 8.2 |
| insulin to glucose (μU | | | | | |
| insulin/mg glucose) | | | | | |
| 24 average active GLP-1 (pM) | 10.9 ± 0.3 | 28.7 ± 0.5 | n.d. | 14.6 ± 0.2 | 29.0 ± 0.5 |

Twenty-four hour average plasma glucose levels were reduced in response to all treatments. Before any treatment, 24 hour average plasma glucose levels were 223.8±34.7 mg/dl in the total virtual patient population and 214.3±37.3 mg/dl in the subpopulation of virtual patients with high GLP-1. When a two-fold increase in the basal GLP-1 release rate was combined with 40% inhibition of DPP-IV, 24 hour glucose was reduced 21% to 175.9±35.9 mg/dl in the total population and 32% to 146.0±27.0 mg/dl in the subpopulation. A three-fold increase in basal GLP-1 release combined with 40% DPP-IV inhibition caused 24 hour glucose levels to fall 28% to 161.0±24.1 mg/dl in the total virtual patient population (the results are not reported for the subpopulation because of high gastric nutrient levels). Treating the total virtual patient population with 40% inhibition of DPP-IV lowered 24 hour average plasma glucose levels 5% to 213.3±33.9 mg/dl, while 100% inhibition lowered 24 hour glucose 24% to 170.3±32.7 mg/dl. The subpopulation of virtual patients with high GLP-1 had greater reductions in 24 hour glucose with 40% (lowered 8% to 198.0±32.7 mg/dl) and 100% (lowered 33% to 144.5±25.6 mg/dl) DPP-IV inhibition.

The ratio of the 24 hour average plasma insulin to the 24 hour average plasma glucose was computed for each treatment of this study. When two-fold increased basal GLP-1 release was combined with 40% DPP-IV inhibition, the ratio of 24 hour insulin to glucose increased 60% in the total population and 94% in the subpopulation. Three-fold increased basal GLP-1 release combined with 40% DPP-IV inhibition raised the ratio 83% in the total population. When DPP-IV was inhibited 40% as a monotherapy, the ratio increased only 10% in the total population and 15% in the subpopulation of virtual patients with high GLP-1. 100% DPP-IV inhibition increased the ratios 68% and 95% in the total population and the subpopulation, respectively.

As expected, 24 hour average active GLP-1 levels increased in response to all treatments. Twenty-four hour average active GLP-1 levels were 8.2±2.0 pM in the total virtual patient population and 10.9±0.3 pM in the subpopulation of virtual patients with high GLP-1. When two-fold increased basal GLP-1 release was combined with 40% DPP-IV inhibition, 24 hour active GLP-1 was increased 162% to 21.5±5.3 pM in the total population and 163% to 28.7±0.5 pM in the subpopulation with high GLP-1. Three fold increased basal GLP-1 plus 40% DPP-IV inhibition increased 24 hour active GLP-1 levels 226% to 26.7±0.8 pM. When DPP-IV was inhibited 40% in the absence of any other treatments, 24 hour active GLP-1 increased 34% in the total population and 34% in the subpopulation of virtual patients with increased GLP-1. 100% DPP-IV inhibition increased active GLP-1 levels 166% in both the total virtual patient population (21.7±5.3 pM) and the subpopulation (29.0±0.5 pM).

Cleavage by DPP-IV rapidly inactivates GLP-1 (half life of approximately 90 second in vivo). Since achieving higher levels of active GLP-1 is predicted to have greater efficacy, inhibition of DPP-IV is expected to increase the efficacy of treatment with GLP-1 secretagogues. The combination of GLP-1 secretagogue treatment with DPP-IV inhibition proved particularly efficacious. Combining two- or three-fold increased basal GLP-1 release with 40% DPP-IV inhibition lowered HbA1c an additional 1-2.5% as compared to when the virtual patients were treated with the DPP-IV inhibitor alone. Ahren et al. reported a reduction in glucose that was similar to the combined GLP-1 secretagogue and DPP-IV inhibitor when subjects with type 2 diabetes were treated for four weeks with the DPP-IV inhibitor LAF237 (Ahren, et al. J Clin Endocrinol Metab. 89:2078-84 (2004)). DPP-IV was inhibited significantly more than 40% with LAF237 treatment. DPP-IV was approximately inhibited 80-90% by LAF237 over a twenty-four hour period. As DPP-IV also participates in degrading many other circulating peptides (including those of the immune system), this degree of inhibition puts subjects at risk for adverse events. Ahren et al. reported adverse events (nasopharyngitis, dizziness, headache, pruritis) in twelve out of eighteen patients treated with LAF237 (Ahren, et al. J Clin Endocrinol Metab. 89:2078-84 (2004)). Any inhibitor of DPP-IV, now known in the art or later discovered, may be used in combination with a GLP-1 secretagogue. Exemplary DPP-IV inhibitors include valine pyrrolidide, isoleucine-thiazolidide, 1-[[(3-hydroxy-1-adamantyl)amino]acetyl]-2-cyano-(S)-pyrrolidine (LAF237), 1-[[[2-[(5-cyanopyridin-2-yl)amino]ethyl]amino]acetyl]-2-cyano-(S)-pyrrolidine (NVP DPP728), and (2S)-1-([2S]-2′-amino-3′,3′-dimethylbutanoyl)-pyrrolidine-2-carbonitrile (FE999011). The level of inhibition can be controlled by altering the amount of DPP-IV inhibitor administered to the patient. Appropriate dosing levels can be determined using conventional methods in the pharmaceutical arts.

One application of GLP-1 secretagogue therapeutics would be to combine them with more modest DPP-IV inhibitors, reducing the risk for adverse events. With this approach, active GLP-1 levels can still be elevated to levels required to achieve appropriate reductions in glycemia without interfering with other physiological processes. In the current study, combining 40% DPP-IV inhibition with increased basal GLP-1 release (two-fold or three-fold) was at least as effective as 100% DPP-IV inhibition alone in all virtual patients. Thus preferably an effective amount of DPP-IV inhibitor would decrease DPP-IV activity by less than 100%, more preferably by less than 60%.

E. Methods of Treating Diabetes in Subjects Having Higher Sensitivity to GLP-1 Secretagogues

An aspect of the invention provides methods of alleviating at least one symptom of diabetes in a diabetic subject having elevated secretion of GLP-1, said method comprising administering a therapeutically effective amount of a glucagon-like peptide-1 (GLP-1) secretagogue.

There is a paucity of data describing GLP-1 levels in subjects with type 2 diabetes, particularly in the postprandial state. Vilsboll et al. reported in two different studies that GLP-1 increased 55-85% when a meal was administered (Vilsboll, et al. Diabetes 50:609-13 (2001); Vilsboll, et al. J Clin Endocrinol Metab. 88:4897-903 (2003)), while Ahren et al. (Ahren, et al. J Clin Endocrinol Metab. 89:2078-84 (2004)) reported a 130% postprandial increase. Moreover, the subjects in the report by Ahren et al. had fasting GLP-1 levels that were 50% lower than the fasting levels in the studies by Vilsboll et al. Part of the variability of response in these three studies could be attributed to the fact that the two research groups used different antibodies to measure GLP-1 levels; another factor could be differential responses amongst subjects. The latter possibility was the impetus for including a subpopulation of virtual patients with an increased postprandial GLP-1 release in this study. In general, this subpopulation exhibited greater reductions in glycemia with GLP-1 secretagogue treatment when compared with the total virtual patients population used in this study. These results suggest that GLP-1 secretagogues could be very efficacious in this population.

One key issue to come out of the current work is the observation that the subgroup of virtual patients with an increased ability to release GLP-1 in response to meals had greater reductions in glycemia with all hypothetical treatments (Tables 2 and 3). This subgroup of virtual patients was generated to help represent the range of postprandial GLP-1 responses observed in subjects with type 2 diabetes. There are only a handful of studies that report postprandial GLP-1 levels in this population, and they differ quite a bit in the reported GLP-1 levels. Vilsboll et al (Diabetes 50:609-13 (2001); J Clin Endocrinol Metab. 88:4897-903 (2003) reported a 55-85% rise in GLP-1 with a small meal, while Ahren et al. (J Clin Endocrinol Metab. 89:2078-84 (2004) reported a 130% rise.

The invention also provides methods of assessing elevated secretion of GLP-1 in a subject comprising (a) measuring a fasting GLP-1 level in the subject after a fast, (b) orally administering about 50 g to about 100 g of glucose to the subject, (c) measuring a stimulated GLP-1 level about 20 to about 90 minutes after orally administering the glucose, and (d) diagnosing the subject as having elevated secretion of GLP-1 if the stimulated GLP-1 level is greater than two-fold the fasting GLP-1 level. As used herein, the term “fast” or “fasting” refers to abstaining from food. Preferably the subject fasts for eight hours, more preferably at least ten hours, most preferably at least twelve hour prior to measurement of plasma concentrations. In addition, it is preferred that the subject fasts for no longer than sixteen hours.

The invention may also be used to assess sensitivity to GLP-1 secretagogue therapy. GLP-1 levels can be determined by any method now known or later discovered. For example, GLP-1 levels can be determined as described by Orskov et al. (Diabetes 43:535-539 (1994)) using standards of synthetic GLP-1(7-36) amide (i.e., proglucagon 78-107 amide) and antiserum 89390.

F. Pharmaceutical Compositions

One aspect of the invention provides methods of manufacturing a drug for use in the treatment of diabetes comprising: (a) identifying a compound as a GLP-1 secretagogue and (b) formulating said compound for concurrent administration to a subject with an inhibitor of dipeptidyl peptidase IV activity. The compound can be identified as a GLP-1 secretagogue, and thereby useful in the treatment of diabetes or insulin resistance, by (i) comparing an amount of GLP-1 secretion in the presence of the compound with an amount of GLP-1 secretion in the absence of the compound; and (ii) identifying the compound as useful in the treatment of diabetes when the amount of GLP-1 secretion in the presence of the compound is at least two-fold greater than the amount of GLP-1 secretion in the absence of the compound.

Compounds capable of inducing secretion of GLP-1 can be identified using cell lines such as human NCI-H716 cells, which can be obtained from the American Type Culture Collection (ATCC, Rockville, Md., USA). In an exemplary protocol, cells are grown in suspension in RPMI 1640 supplemented with 10% FBS, 2 mM L-glutamine, 100 IU/ml penicillin and 100 μg/ml streptomycin at 37° C., 5% CO₂. Endocrine differentiation can be enhanced in vitro in NCI-H716 cells grown on an extracellular matrix e.g. by seeding in dishes coated with MATRIGEL®. (Becton Dickinson, Bedford, Mass., USA) two days before experiments. On the day of the experiment, the supernatant is replaced by Krebs-Ringer Bicarbonate Buffer (KRB) containing 0.2% wt/vol BSA with or without the test compound. Supernatants are collected after a two hour incubation at 37° C. with the addition of 50 μg/ml PMSF. The samples can be frozen at −80° C. for subsequent analysis by radioimmunoassay (RIA) of GLP-1. Cells are harvested from the dishes suspended in homogenization buffer (1 N HCl containing 5% (v/v) HCOOH, 1% (v/v) trifluoroacetic acid (TFA), and 1% (v/v) NaCl) and sonicated for 15 s. Concentrations of GLP-1 (Total, i.e., 7-36 amide or 9-36 amide) are measured using a commercial RIA kit (Linco Research Inc., St. Charles, Mo., USA).

Compounds capable of inhibiting DPP-IV can be identified using DPP-IV, obtained from porcine kidneys. The DPP-IV, dissolved in a reaction buffer solution (50 mM Tris-HCl, pH 7.4, 0.1% BSA), is combined with a test compound and incubated at room temperature for 20 minutes. Twenty-five microliters of a solution in which Gly-Pro-p-nitroanilide is dissolved at 2 mM is added (final concentration, 0.33 mM) to start the enzymatic reaction. The reaction is stopped after 20 minutes by the addition of phosphoric acid. The absorbance at 405 nm is measured to determine the percent inhibition of the enzyme reaction.

The treatment modes of this study were chosen to represent different possible avenues for increasing GLP-1 release. GLP-1 is primarily released in response to the appearance of nutrients in the small intestines (Schirra, et al. J Clin Invest. 97:92-103 (1996); Rocca, et al. Endocrinology 142:1148-55 (2001); Thomsen, et al. Am J Clin Nutr. 69:1135-43 (1999)), although a basal level of GLP-1 is also secreted in the fasted state. An increase in the basal rate of GLP-1 release could be achieved by a compound that could be delivered humorally or via the lumen of the intestines. Intestinal delivery would require persistence of a signal, implying that the signal would need to have a high residence time in the intestinal lumen. Advances in drug delivery may make this route feasible. Amplifying GLP-1 release to increase postprandial excursions could be achieved by administering an agent with a meal. Several nutrients are potent secretagogues for GLP-1, including oleic acid (Rocca, et al. Endocrinology 142:1148-55 (2001)) and glucose (Schirra, et al. J Clin Invest. 97:92-103 (1996)). Also, pharmaceutical agents such as the biguanides have been shown to have the ability to stimulate GLP-1 release (Yasuda, et al. Biochem Biophys Res Commun. 298:779-84 (2002)). Formulating meals that include appropriate amounts of these substances could potentiate the postprandial GLP-1 release rate. However, including a GLP-1 secretagogue with a meal subjects the agent to the reduction of gastric emptying that is imposed by elevated levels of GLP-1. This would serve to restrict the delivery of the stimulatory signal, and, ultimately, would restrict the increase in GLP-1 levels. Thus, delivery of GLP-1 secretagogues with a meal may not provide the maximum ability to elevate GLP-1 levels. A GLP-1 secretagogue that gives a constant stimulus would be advantageous in that it would be able to overcome the regulation provided by the GLP-1-driven restricted gastric emptying. Moreover, it would increase GLP-1 levels in the fasted state as well as the fed state. As discussed above, the greater the increase in 24 hour active GLP-1 levels, the greater the reduction in HbA1c (FIG. 2).

Compounds useful in this invention are administered to a diabetic and/or insulin-resistant subject in a therapeutically effective dose by a medically acceptable route of administration. The dosage range adopted will depend on the route of administration and on the age, weight and condition of the subject being treated. Regardless of the route of administration selected, the GLP-1 secretagogue and the DPP-IV inhibitor are formulated into pharmaceutically acceptable unit dosage forms by conventional methods known to the pharmaceutical art. An effective but nontoxic quantity of the GLP-1 secretagogue and of the DPP-IV inhibitor are employed in the treatment. The GLP-1 secretaoguge and the DPP-IV inhibitor may be concurrently administered enterally and/or parenterally in admixture or separately. Parenteral administration includes subcutaneous, intramuscular, intradermal, intravenous, injection directly into the joint and other administrative methods known in the art. Enteral administration includes tablets, sustained release tablets, enteric coated tablets, capsules, sustained release capsules, enteric coated capsules, pills, powders, granules, solutions, and the like.

Various delivery systems are known and can be used to administer a composition of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Oral formulations for use in the present invention preferably are prepared so as to provide a targeted and controlled release of the GLP-1 secretagogue in the intestinal lumen with minimal or no release in the stomach. Preferably, the GLP-1 secretagogue is associated in a slow release formulation, e.g., a tablet, so as to provide delayed or controlled release of the GLP-1 secretagogue in the region of the intestine having a pH relatively near the neutral range. For example, the drug is formulated with a delayed drug release dependent on transit time, amount of hydration or the presence or absence of other physiochemical variables.

The pharmaceutical compositions of the present invention comprise one or more excipients and/or carriers known in the pharmaceutical arts which delay the release of the GLP-1 secretagogue at the desired target in the gastrointestinal tract, i.e. after exiting the stomach. In addition, the release of the GLP-1 secretagogue may be immediate, i.e., the release may be delayed until the drug reaches the targeted site, but than the release is immediate upon entry to the target site. On the other hand, the present invention contemplates sustained release formulation, wherein the pharmaceutical composition, besides comprising the GLP-1 secretagogue compound, and carrier or excipient targeted for a specific site in the body, may also contain a sustained release carrier or excipient, e.g., sustained release polymer, to prolong the release thereof over a period of time. The pharmaceutical composition may comprise one or more sustained or controlled release excipients or carriers, such that a slow or sustained release of the GLP-1 secretagogue is achieved. A wide variety of suitable excipients are known in the art.

pH sensitive materials have been widely used as enteric coatings to encapsulate and/or protect active ingredients during transit through the stomach, and then release the agent shortly after entering the small intestine. Exemplary delivery systems utilizing pH-sensitive coatings have been described in publications such as WO 9001329 and U.S. Pat. Nos. 4,910,021, 5,175,003, 5,484,610, 6,068,859, 6,103,865 and 6,228,396. pH sensitive osmotic bursting devices have described for dispensing drugs to certain pH regions of the gastrointestinal tract. Exemplary systems are described in U.S. Pat. Nos. 4,503,030, 5,609,590 and 5,358,502. There are also hybrid systems which combine pH-sensitive materials and osmotic delivery systems. See, for example, U.S. Pat. Nos. 4,578,075, 4,681,583, 4,851,231, 4,096,238, 4,503,030, 4,522,625, and 4,587,117.

GLP-1 secretagogue treatment has potential in reducing glycemia in subjects with type 2 diabetes. The study described in this report indicates that the efficacy of GLP-1 secretagogues is comparable or better than what is currently being reported in clinical studies. Moreover, combining GLP-1 secretagogue treatment with modest inhibition of DPP-IV could reduce glucose levels and avoid some of the adverse events associated with more severe DPP-IV inhibition (nasopharyngitis, dizziness, headache, pruritis). The most practical application of the results of this study are in the formulation of a constant stimulus for GLP-1 release, as this would avoid the negative feedback loop driven by GLP-1-induced restricted gastric emptying.

While the above is a complete description of possible embodiments of the invention, various alternatives, modifications and equivalents may be used to which the invention is equally applicable. Therefore, the above description should be viewed as only a few possible embodiments of the present invention, the boundaries of which is appropriately defined by the metes and bounds of the following claims.

What is claimed is:

1. A method of alleviating at least one symptom of diabetes comprising concurrently administering a therapeutically effective amount of a glucagon-like peptide-1 (GLP-1) secretagogue and a therapeutically effective amount of an inhibitor of dipeptidyl peptidase IV (DPP-IV) activity to a subject having diabetes.

2. The method of claim 1, wherein the subject has type 2 diabetes.

3. The method of claim 1, wherein the symptom of diabetes is selected from the group consisting of twenty-four hour average plasma glucose levels, twenty-four hour average plasma insulin levels.

4. The method of claim 1, wherein the symptom of diabetes is elevated glycosylated hemoglobin (HbA1c).

5. The method of claim 4, wherein concurrent administration of the GLP-1 secretagogue and the DPP-IV inhibitor decreases the absolute value of the subject's HbA1c by at least 1.0%.

6. The method of claim 5, wherein concurrent administration of the GLP-1 secretagogue and the DPP-IV inhibitor decreases the absolute value of the subject's HbA1c by at least 1.2%.

7. The method of claim 6, wherein concurrent administration of the GLP-1 secretagogue and the DPP-IV inhibitor decreases the absolute value of the subject's HbA1c by at least 1.6%.

8. The method of claim 1, wherein the symptom of diabetes is elevated twenty-four hour average blood glucose concentration.

9. The method of claim 8, wherein concurrent administration of the GLP-1 secretagogue and the DPP-IV inhibitor decrease the subject's twenty-four hour average blood glucose concentration by at least 21%.

10. The method of claim 9, wherein concurrent administration of the GLP-1 secretagogue and the DPP-IV inhibitor decrease the subject's twenty-four hour average blood glucose concentration by at least 28%.

11. The method of claim 10, wherein concurrent administration of the GLP-1 secretagogue and the DPP-IV inhibitor decreases the subject's twenty-four hour average blood glucose concentration by at least 32%.

12. The method of claim 1, wherein the GLP-1 secretagogue is administered parenterally.

13. The method of claim 1, wherein the GLP-1 secretagogue is administered enterally.

14. The method of claim 13, wherein the GLP-1 secretagogue is administered via the lumen of the intestines.

15. The method of claim 1, wherein the GLP-1 secretagogue increase basal GLP-1 release by at least two-fold.

16. The method of claim 15, wherein the GLP-1 secretagogue increases basal GLP-1 release by at least three-fold.

17. The method of claim 1, wherein the subject has elevated secretion of GLP-1 prior to administration of the GLP-1 secretagogue.

18. The method of claim 1, wherein the therapeutically effective amount of the DPP-IV inhibitor decreases DPP-IV activity by at least 40%.

19. The method of claim 1, wherein the therapeutically effective amount of the DPP-IV inhibitor decreases DPP-IV activity by no greater than 60%.

20. The method of claim 1, wherein the GLP-1 secretagogue is selected from the group consisting of a fatty acid, a carbohydrate, and a biguanide.

21. The method of claim 20, wherein the fatty acid is oleic acid.

22. The method of claim 20, wherein the biguanide is metformin.

23. The method of claim 1, wherein the DPP-IV inhibitor is selected from the group consisting of valine pyrrolidide, isoleucine-thiazolidide, 1-[[(3-hydroxy-1-adamantyl)amino]acetyl]-2-cyano-(S)-pyrrolidine (LAF237), 1-[[[2-[(5-cyanopyridin-2-yl)amino]ethyl]amino]acetyl]-2-cyano-(S)-pyrrolidine (NVP DPP728), and (2S)-1-([2S]-2′-amino-3′,3′-dimethylbutanoyl)-pyrrolidine-2-carbonitrile (FE999011)

24. A method of alleviating at least one symptom of diabetes in a diabetic subject having elevated secretion of GLP-1, said method comprising administering a therapeutically effective amount of a glucagon-like peptide-1 (GLP-1) secretagogue.

25. The method of claim 24, wherein the subject has type 2 diabetes.

26. The method of claim 24, wherein the symptom of diabetes is selected from the group consisting of twenty-four hour average plasma glucose levels, twenty-four hour average plasma insulin levels.

27. The method of claim 24, wherein the symptom of diabetes is elevated glycosylated hemoglobin (HbA1c).

28. The method of claim 27, wherein administration of the GLP-1 secretagogue decreases the absolute value of the subject's HbA1c by at least 1.0%

29. The method of claim 28, wherein administration of the GLP-1 secretagogue decreases the absolute value of the subject's HbA1c by at least 1.6%.

30. The method of claim 29, wherein administration of the GLP-1 secretagogue decreases the absolute value of the subject's HbA1c by at least 1.9%.

31. The method of claim 24, wherein the symptom of diabetes is elevated twenty-four hour average blood glucose concentration.

32. The method of claim 31, wherein administration of the GLP-1 secretagogue decreases the subject's twenty-four hour average blood glucose concentration by at least 18%.

33. The method of claim 32, wherein administration of the GLP-1 secretagogue and decreases the subject's twenty-four hour average blood glucose concentration by at least 27%.

34. The method of claim 33, wherein administration of the GLP-1 secretagogue decreases the subject's twenty-four hour average blood glucose concentration by at least 35%.

35. The method of claim 24, wherein the GLP-1 secretagogue increase basal GLP-1 release by at least two-fold.

36. The method of claim 35, wherein the GLP-1 secretagogue increases basal GLP-1 release by at least three-fold.

37. The method of claim 24, wherein the GLP-1 secretagogue increases postprandial GLP-1 release by at least two-fold.

38. The method of claim 37, wherein the GLP-1 secretagogue increases postprandial GLP-1 release by at least three-fold.

39. The method of claim 24, wherein the GLP-1 secretagogue is selected from the group consisting of a fatty acid, a carbohydrate, and a biguanide.

40. The method of claim 39, wherein the GLP-1 secretagogue is oleic acid.

41. The method of claim 39, wherein the GLP-1 secretagogue is metformin.

42. A method of assessing elevated secretion of GLP-1 in a subject comprising:
(a) measuring a fasting GLP-1 level in the subject after a fast;
(b) orally administering about 50 g to about 100 g of glucose to the subject;
(c) measuring a stimulated GLP-1 level about 20 to about 90 minutes after orally administering the glucose; and
(d) diagnosing the subject as having elevated secretion of GLP-1 if the stimulated GLP-1 level is greater than two-fold the fasting GLP-1 level.

43. A method of assessing sensitivity to GLP-1 secretagogue therapy comprising:
(a) measuring a fasting GLP-1 level in a subject after a fast;
(b) orally administering about 50 g to about 100 g of glucose to the subject;
(c) measuring a stimulated GLP-1 level about 20 to about 90 minutes after orally administering the glucose; and
(d) identifying the subject as sensitive to GLP-1 secretagogue therapy if the stimulated GLP-1 level is greater than two-fold the fasting GLP-1 level.

44. A method of manufacturing a drug for use in the treatment of diabetes comprising:
(a) identifying a compound as useful in the treatment of diabetes by:
(i) comparing an amount of GLP-1 secretion in the presence of the compound with an amount of GLP-1 secretion in the absence of the compound; and
(ii) identifying the compound as useful in the treatment of diabetes when the amount of GLP-1 secretion in the presence of the compound is at least two-fold greater than the amount of GLP-1 secretion in the absence of the compound; and
(b) formulating said compound for concurrent administration to a subject with an inhibitor of dipeptidyl peptidase IV activity.

45. The method of claim 44, wherein GLP-1 secretion is measured by a process comprising the step(s) of:
a) incubating human NCI-H716 cells in the presence or absence of the compound;
b) collecting a cell supernatant from the incubated cells; and
c) measuring the amount of GLP-1 in the supernatant by radioimmunoassay.

46. A pharmaceutical composition for treating diabetes comprising:
a) a therapeutically effective amount of a glucagon-like peptide-1 (GLP-1) secretagogue; and
b) pharmaceutically acceptable carrier targeted that delays release of the GLP-1 secretagogue until after exiting the stomach.

47. The pharmaceutical composition of claim 46, wherein the pharmaceutically acceptable carrier is a pH sensitive material.

48. A package comprising:
a) a therapeutically effective amount of a glucagon-like peptide-1 (GLP-1) secretagogue
c) a therapeutically effective amount of an inhibitor of dipeptidyl peptidase IV activity; and
b) a label with instructions for concurrently administering the secretagogue and the inhibitor for treating diabetes.
